(19) United States
(12) Patent Application Publication
(10) Pub. No.: US 2016/0304837 A1
(43) Pub. Date: Oct. 20, 2016

(54) Method of ex vivo expanding hematopoietic stem/ progenitor cells and the composition produced thereby

(71) Applicant: Taiwan Advance Bio-Pharmaceutical, New Taipei City (TW)

(72) Inventors: Chi-Hung Huang, Taoyuan City (TW); Ting-Yun Liu, Taipei City (TW); Yu-Lin Chen, New Taipei City (TW)

(21) Appl. No.: 15/130,421

(22) Filed: Apr. 15, 2016

(30) Foreign Application Priority Data
Apr. 16, 2015 (TW) 104112178

(51) Int. Cl. C12N 5/0789

(52) U.S. Cl. CPC C12N 5/0647

(57) ABSTRACT

The present invention relates to a process for rapidly ex vivo expanding and harvesting high-purity of hematopoietic stem/progenitor cells and the pharmaceutical composition comprising the same. The process of the present invention is characterized by: an overnight culture of mononuclear cells isolated by density gradient centrifugation; and subsequent purification and ex vivo expansion of high-purity hematopoietic stem/progenitor cells. The prepared hematopoietic stem/progenitor cells comprise high percentage of clinically effective hematopoietic stem/progenitor cells (the CD34⁺ CD38⁻ cells), and still maintain high viability and effective differential activity after cryopreservation and thawing processes. Besides, for the manufacturing method of the present invention does not use components of animal origin, the harvested hematopoietic stem/progenitor cells can be directly used in clinical applications.

### BACKGROUND OF THE INVENTION

1. Technical Field of the Invention

The present invention relates to a process for rapidly ex vivo expanding and harvesting high-purity of hematopoietic stem/progenitor cells and the composition comprising the same. Especially, the present invention relates to a method of purifying and ex vivo expanding hematopoietic stem/progenitor cells after overnight culture of mononuclear cells which are isolated by density gradient centrifugation. The prepared hematopoietic stem/progenitor cells comprise high percentage of clinically effective hematopoietic stem/progenitor cells, the CD34⁺ CD38⁻ cells. For there are no components of animal origin used in the manufacturing method of present invention, the harvested hematopoietic stem/progenitor cells can be directly used in clinical applications.

2. Background

Hematopoietic stem cells (HSCs), which are the predecessors of mature blood cells, have abilities to self-renew and to differentiate into any type of blood cells (Blood, Vol. 89, No. 12, 1997: pp 4337-4347). Until now, Hematopoietic stem cell transplantation has already used in the treatment of hematologic diseases and congenital diseases. Generally, bone marrow, peripheral blood and umbilical cord blood are promising sources of hematopoietic stem cells for clinical use. Due to the pain and uncomfortable feeling occurred in the bone marrow harvesting procedure, people prefer to select peripheral blood as the source of hematopoietic stem cells. However, it is hard to find a suitable match from an unrelated donor, in present, peripheral blood is replaced by umbilical cord blood to be the source of hematopoietic stem cells for HSC transplantation.

Clinical studies have confirmed that the survival and success rate after CD34⁺ cell transplantation are directly proportional to the amount of CD34⁺ cells ready for transplantation.

CD34 is a surface antigen of human hematopoietic stem cells, usually used as a marker for hematopoietic stem cells. The clinical study has proved that the success and survival rate after hematopoietic stem cell transplantation is associated with the amounts of transplanted CD34⁺ cells. Further study found that the cells expressing CD34 but not CD38 (so called as CD34⁺CD38⁻ cells) are the hematopoietic stem cells truly having therapeutic affectivities.

Although umbilical cord blood hematopoietic stem cell transplantation has a great effect on clinical application, the number of hematopoietic stem cells for transplantation in clinical, for instance, the number of total nuclear cells (TNC) is requested to be at least 2×10⁷ cells/kg, and the amount of CD34⁺ cells should be 2×10⁵ cells/kg. Our issue is that: the amount of hematopoietic stem cell from umbilical cord blood is rare. Therefore, there are many researches that work for hematopoietic stem cells ex vivo expansion.

There are lots of expanding methods for hematopoietic stem cells described in prior arts. For example, U.S. patent application Ser. No. 11/255,191 discloses a method for ex vivo expanding and analyzing hematopoietic stem cells, comprising the use of effective amounts of various cytokines. The application further provides a kit for identifying the isolated hematopoietic stem cells. Japan patent application No. JP2010188594 relates to a preparation process of hematopoietic stem cells, comprising adding an expanding agent to the medium for hematopoietic stem cells to amplify the number of cultured cells. Chinese patent application No. CN2012100087227 provides an ex vivo expanding method for hematopoietic stem cells by using a soluble fusion protein D111-RGD (hD111-RGD), which is a Notch ligand for targeting vascular endothelial cells. In addition of promoting the hematopoietic stem cell proliferation, Taiwan patent application No. 098115726 discloses a method and system for isolating, ex vivo expanding and harvesting hematopoietic stem cells, which is characterized by quick isolation and expansion of hematopoietic stem cells to enhance the harvesting efficiency.

The current ex vivo expanding methods for hematopoietic stem cells mainly comprise the addition of various substances, such as cytokines, compounds and/or recombinant proteins, to the culture medium to promote the proliferation of hematopoietic stem cells. However, the culture time of hematopoietic stem cells is longer than 7 days, even more than 2 weeks, which makes impossible to obtain the clinically effective hematopoietic stem cell population (CD34⁺ CD38⁻ cells) in a short period. Since the resource of hematopoietic stem cells is extremely rare and fragile, how to effectively enhance the recovery rate and yield of hematopoietic stem cells, and to preserve the hardly acquired hematopoietic stem cells, become the key points of ex vivo expanding hematopoietic stem cells.

### SUMMARY OF INVENTION

Accordingly, in one aspect, the present invention provide a rapid ex vivo expanding method for high-purity and instantly available hematopoietic stem/progenitor cells, comprising enhancing the recovery of hematopoietic stem/progenitor cells by the steps of: isolating high-purity mononuclear cells by gradient density gradient centrifugation; culturing the high-purity mononuclear cells overnight to restore cell activity; and purifying high-purity hematopoietic stem/progenitor cells.

In some embodiments, the ex vivo expanding method comprises: thawing the blood containing hematopoietic stem/progenitor cells, and isolating high-purity mononuclear cells by gradient density gradient centrifugation; culturing the high-purity mononuclear cells overnight then purifying high-purity hematopoietic stem/progenitor cells; incubating the high-purity hematopoietic stem/progenitor cells in IMDM/5% HABS medium supplemented with cytokines and TAT-HOXB4 for 4-7 days; and harvesting the hematopoietic stem/progenitor cells. In one embodiment, the overnight culture comprises incubating the high-purity mononuclear cells in a medium at a cell density of 5×10⁵˜6×10⁶ cells/mL for 16˜18 hours. In another embodiment, the blood is umbilical cord blood or peripheral blood.

In one embodiment, the high-purity hematopoietic stem/progenitor cells are incubated in IMDM/5% HABS medium supplemented with cytokines and TAT-HOXB4 at 1×10⁴˜5×10⁵ cells/mL cell density. In another embodiment, the cytokines are IL-3, IL-6, SCF, FLT-3L or TPO.

In another aspect, the method of present invention further comprises the step of: freezing the prepared hematopoietic stem/progenitor cells with a cryoprotectant containing 24˜80% Albuminar®-25 and 20% CrySure-DEX40 (containing 6˜20% human albumin).

In a further aspect, the present invention provide a hematopoietic stem/progenitor cell composition prepared in accordance with the method of the present invention, comprising 15˜40% of clinically effective hematopoietic stem/progenitor cells (CD34⁺CD38⁻ cells). Preferably, the composition of the present invention comprises 25˜30% of clinically effective hematopoietic stem/progenitor cells (CD34⁺CD38⁻ cells).

### BRIEF DESCRIPTION OF THE DRAWINGS

FIG. 1 shows the recovery of hematopoietic stem/progenitor cells in various purification procedures by flow cytometry.

FIG. 2 shows the fold change of the hematopoietic stem/progenitor cells were cultured in three group of medium with different components. FIG. 2A shows the fold change of total nuclear cell numbers. FIG. 2B shows the fold change of CD34⁺ cell numbers.

FIG. 3 shows the expanding ratio of hematopoietic stem/progenitor cells were cultured in various medium with different components at indicated of cell density by flow cytometry.

FIG. 4 shows the effect of different cell densities on the expansion of hematopoietic stem/progenitor cells. FIG. 4A is the fold change of total nuclear cell numbers. FIG. 4B shows the fold change of CD34⁺ cell numbers.

FIG. 5 shows the analysis of different cryoprotectants for their effects on the stability of hematopoietic stem/progenitor cells. FIG. 5A is analysis of cell survival rate. FIG. 5B is the ratio of hematopoietic stem/progenitor cells by flow cytometry.

### DETAILED DESCRIPTION OF THE INVENTION

The other characteristics and advantages of the present invention will be further illustrated and described in the following examples. The examples described herein are using for illustrations, not for limitations of the invention.

### Example 1

### Purification and Recovery of Hematopoietic Stem/Progenitor Cells

To be one of the technical features of present invention, the recovery ratio of hematopoietic stem/progenitor cells is increased by purification and overnight culture of monocytes. Therefore, there were two experimental groups of included in the example. In one of the groups, umbilical cord bloods or peripheral bloods containing hematopoietic stem/progenitor cells were thawed and used for the purification of monocytes by density gradient centrifugation with Ficoll-Paque after the removal of DMSO by centrifugation. The hematopoietic stem/progenitor cells are isolated at the same day. Processes of hematopoietic stem/progenitor cells purification comprised the following steps: first, the purified monocytes described above were suspended in 300 μl 1×PBS or normal saline with 0.5% human albumin then mixed with 100 μl FcR blocking buffer and 100 μl CD34 magnetic microbeads and rotated at 4° C. for 30 mins. Subsequently, the microbeads were diluted with 5 ml 4° C. 1×PBS or normal saline with 0.5% human albumin, and then centrifuged at 300 g at 4-12° C. for 10 mins. Resuspended microbeads in 3 ml 4° C. 1×PBS or normal saline with 0.5% human albumin were loaded onto a column which is placed in the magnetic field and washed with 5 ml 4° C. 1×PBS or normal saline with 0.5% human albumin for three times. After removing the column from the magnetic field, the magnetically retained CD34⁺ cells were eluted as the positively selected cell fraction.

In another group, the monocytes were obtained with or without red blood cell lysis by red blood cell lysis buffer using density gradient centrifugation. The hematopoietic stem/progenitor cells were purified at next day after culturing the monocytes in IMDM/5% HABS medium at 5×10⁵-6×10⁶ cells/mL density at 37° C. and 5% CO2 incubator for 16-18 hours.

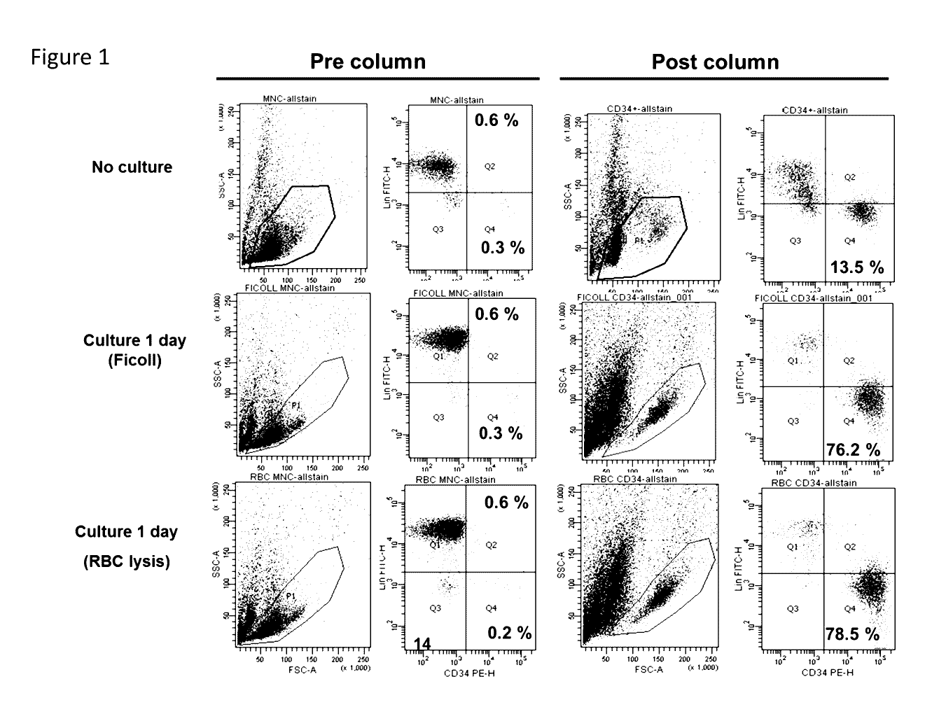

The results of monocytes colony and purified hematopoietic stem/progenitor cells of each groups described above were measured by flow cytometry. As shown in FIG. 1, we found no difference in the purity of monocytes between groups, while the purity of hematopoietic stem/progenitor cells of the groups of monocular cells over night incubation were increased more than 76.2% as compared to the groups of immediate purified hematopoietic stem/progenitor cells thawed from blood (13.5%). The results suggest that the method of monocular cells overnight incubation increased the recovery of hematopoietic stem/progenitor cells by restored the activity of monocular cells that injured by cell thawing.

### Example 2

### Ex Vivo Expansion of Hematopoietic Stem/Progenitor Cells

In addition to modified purification, the compositions of medium and the cell density of cell culture are two key factors that affect ex vivo expansion rate of hematopoietic stem/progenitor cells. Therefore, in the present invention, we focused on these key factors. The high-purity hematopoietic stem/progenitor cells obtained in the Example 1 were cultured in IMDM/5% HABS medium with various cytokines for 4 days, then subjected to analysis of cell proliferation rate. In this experiment, we selected IMDM/5% HABS medium supplemented with different compositions as following:

(1) Composition 1: with 5 ng/mL IL-3, 10 ng/mL IL-6, 50 ng/mL SCF, 20 ng/mL FLT-3L and 15 nM TAT-HOXB4.
(2) Composition 2: with 5 ng/mL IL-3, 10 ng/mL IL-6, 100 ng/mL SCF, 20 ng/mL FLT-3L and 15 nM TAT-HOXB4.
   (3) Composition 3: with 5 ng/mL IL-3, 10 ng/mL IL-6, 100 ng/mL SCF, 20 ng/mL FLT-3L, 25 ng/mL TPO and 15 nM TAT-HOXB4.

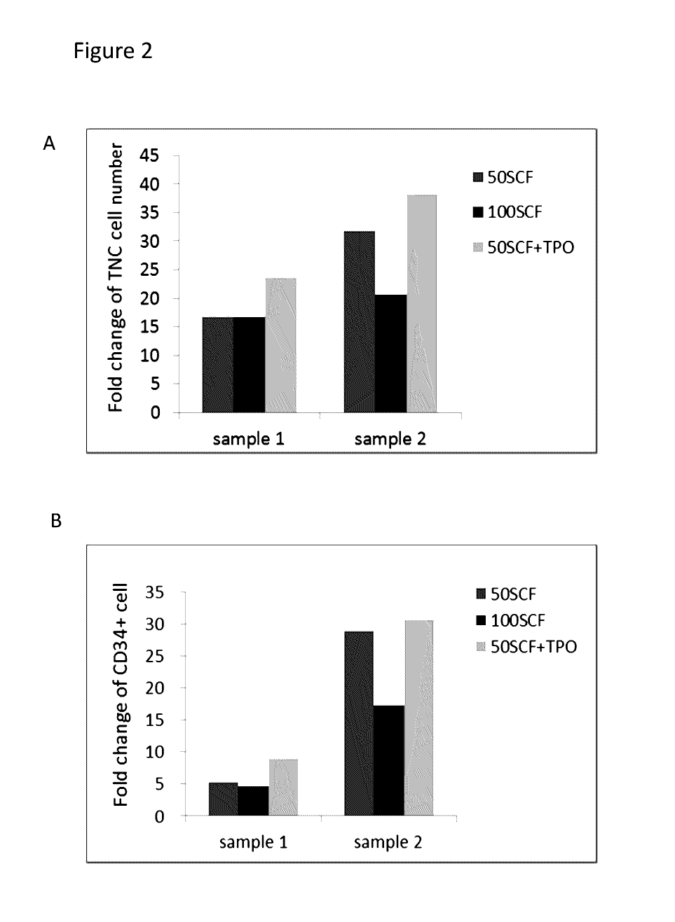

As shown in FIG. 2, the results of the fold change of total nuclear cells and CD34⁺ cells were both increased in cell line 1 and cell line 2 in the groups. The fold change of the composition 3 was the most significant group among the groups. These results suggest that the composition and ratio of cytokines extremely affect expansion of hematopoietic stem/progenitor cells.

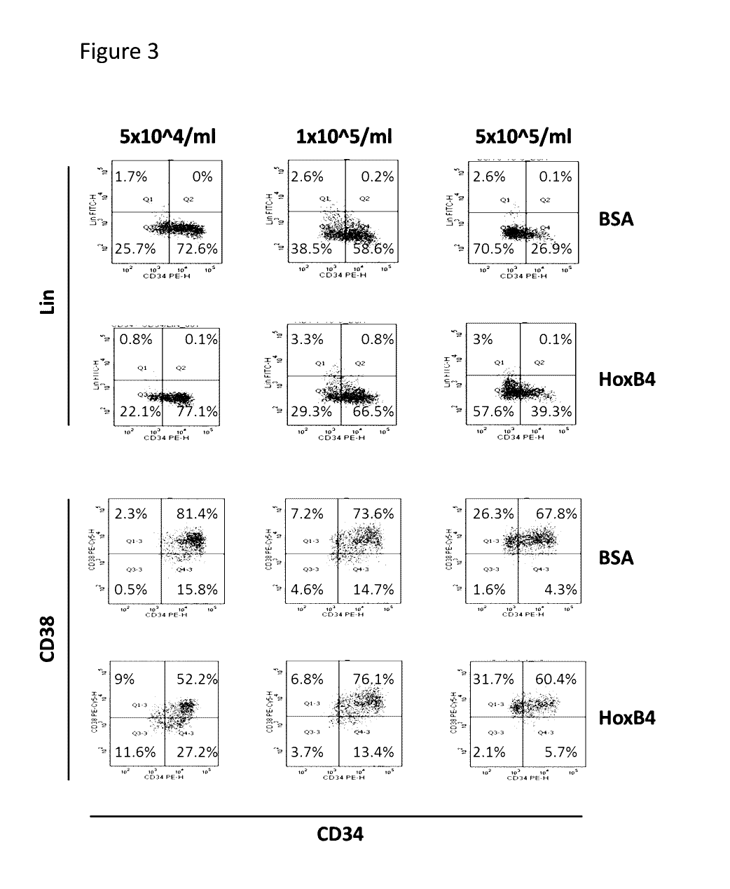

Besides the effect of the medium compositions on hematopoietic stem/progenitor cell proliferation, the culture density is also one of the key factors of cell expansion. In this study, the high-purity hematopoietic stem/progenitor cells were seeded with indicated cell densities (5×10⁴, 1×10⁵, 5×10⁵ cells/mL) in IMDM/5% HABS medium supplemented with 5 ng/mL IL-3, 10 ng/mL IL-6, 100 ng/mL SCF, 20 ng/mL FLT-3L, 25 ng/mL TPO and 0.1% BSA or with Composition 3 described above for 4 days, and then measured the cell numbers by flow cytometry, shown as Table 1 and FIG. 3.

TABLE 1

| Cell density | | 5 × 10\hat{ }4/ml | 1 × 10\hat{ }5/ml | 5 × 10\hat{ }5/ml |
|---|---|---|---|---|
| Initial purity(1322) | | 97.2% | | |
| Initial CD34+/CD38− | | 7% | | |
| Initial cell number | | 2.5 × | 2.5 × | 2.5 × |
| | | 10\hat{ }5/5 ml | 10\hat{ }5/2.5 m | 10\hat{ }5/0.5 m |
| BSA | CD34+(%) | 72.5 | 58.8 | 27 |
| | CD34+/CD38−(%) | 15.8 | 14.7 | 4.3 |
| | TNC number(10\hat{ }6) | 3.4 | 3.28 | 3.07 |
| | HSC number(10\hat{ }5) | 24 | 19.1 | 8.29 |
| | TNC expansion fold | 13.6 | 13.2 | 1.23 |
| | HSC expansion fold | 9.88 | 7.86 | 3.41 |
| HB4 | CD34+(%) | 77.2 | 67.3 | 39.4 |
| | CD34+/CD38−(%) | 27.2 | 13.4 | 5.7 |
| | TNC number(10\hat{ }6) | 4.18 | 3.86 | 3.21 |
| | HSC number(10\hat{ }5) | 32.85 | 26 | 12.65 |
| | TNC expansion fold | 16.72 | 15.44 | 12.84 |
| | HSC expansion fold | 13.52 | 10.7 | 5.19 |

As the results shown in Table 1 and FIG. 3, although cells could proliferate in different compositions and different cell density, the proportions of cell colony were not the same. Firstly, as comparing to different culture medium, there was no difference to last experiment. The ratio of effective hematopoietic stem/progenitor cells which were cultured in the medium with Composition 3 for 4 days was significantly higher than the group of BSA.

Moreover, compared with cell density, we observed that the ratio of effective hematopoietic stem/progenitor cells which were cultured at 5×10⁴ cells/mL cell density for 4 days was 72.5-77.2%. Together, the colony of pervious hematopoietic stem/progenitor cells was only 7%, it could be expand to 27.2% after in IMDM/5% HABS medium supplemented with Composition 3 at 5×10⁴ cells/mL cell density and the fold of TNC was also increased to 13.52.

Further, we based on 5×10⁴ cells/mL cell density to down regulation cell density to find the best culture condition of hematopoietic stem/progenitor cells, the cell density were as following:

(1) Cell density group 1: culture at 1×10⁴ cells/mL cell density to 7^{th} day.
(2) Cell density group 2: culture at 5×10⁴ cells/mL cell density for 3 days then culture at 1.5×10⁴ cells/mL cell density to 7^{th} day.
(3) Cell density group 3: culture at 5×10⁴ cells/mL cell density for 3 days then culture at 3×10⁴ cells/mL cell density to 7^{th} day.
(4) Cell density group 4: culture at 5×10⁴ cells/mL cell density to 7^{th} day.

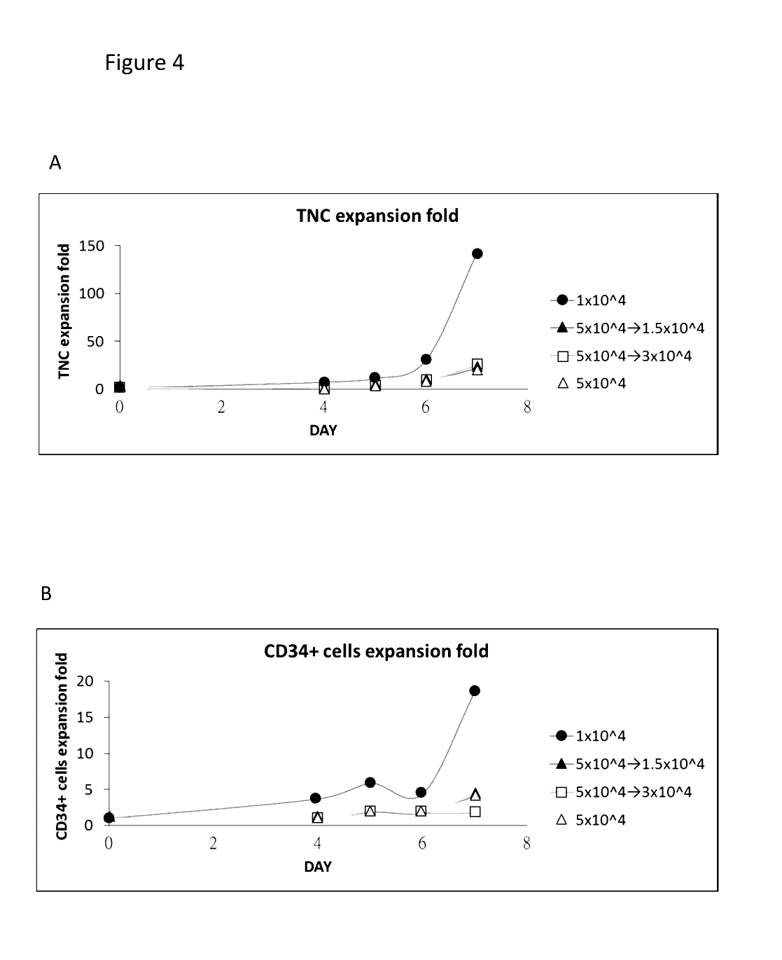

As shown in FIG. 4, we observed that the fold of TNC expansion and CD34⁺ cells of Cell density group 1 was significantly expanded to 141.5 and 18.5, respectively. This result indicated that cell density is one of important condition of effective hematopoietic stem/progenitor cells.

### Example 3

### Cell Freezing of Ex Vivo Expansion of Hematopoietic Stem/Progenitor Cells

According to experiments above have already expanding cell ex vivo efficiently. However, how to remain the cell survival rate after cell freezing and thawing was also important. Therefore, in this experiment, we examined with 3 sort of cell cryoprotectants as follow:

(1) Formula 1: contains 80% Albuminar®-25 and 20% Cry Sure-DEX40 (containing 20% human albumin).
(2) Formula 2: contains 48% Albuminar®-25, 20% CrySure-DEX40 and saline (containing 12% human albumin).
(3) Formula 3: contains 24% Albuminar®-25, 20% CrySure-DEX40 and saline (containing 6% human albumin).

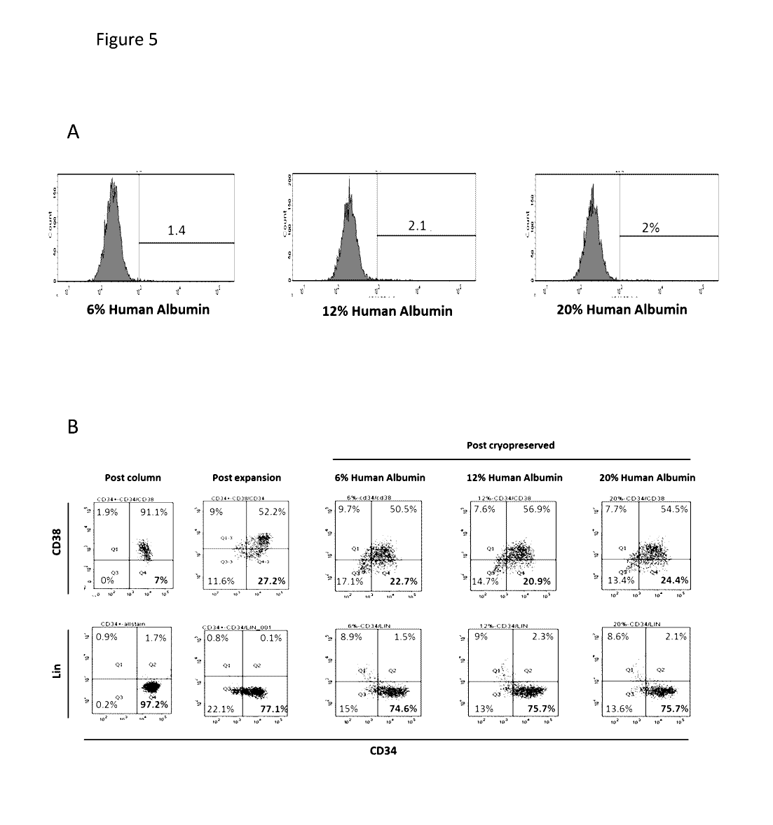

The hematopoietic stem/progenitor cells which were harvested form the 4-day culture at 1×10⁴ cells/mL cell density were frozen with the cell cryoprotectant Formulal-3 as described above, and then stored in BioArchive system for 1 month. After that, the cell survival rate in different groups were measured, and the results are shown in FIG. 5. According to FIG. 5A, the survival rate after cell thawing have no different in groups. We further measured the cell colony of hematopoietic stem/progenitor cells by flow cytometry. Shown as FIG. 5B, the proportion of effective hematopoietic stem/progenitor cells which were freeze with Formula 3 was better than others after thawing. These results suggest that use suitable cryoprotectant could remain high proportion of purity and survival rate of hematopoietic stem/progenitor cells.

In conclusions, we demonstrated the method of the present invention could efficiently expand high-purity hematopoietic stem/progenitor cells and harvest amount of clinical effectiveness of hematopoietic stem/progenitor cells in few days (4˜7 days) by culturing in indicated compositions of medium at indicated cell densities. At last, preserving the proportion of clinical effectiveness of hematopoietic stem/progenitor cells and cell survival rate by cell freezing with indicated cryoprotectants.

Besides, for the method of the present invention including cell purification, ex vivo expansion and cell freezing does not use components of animal origin, the compositions prepared by the method of the present invention could be directly used in clinical application.

1. A method for rapid ex vivo expanding high-purity and instantly available hematopoietic stem/progenitor cells, comprising:
thawing a blood containing hematopoietic stem/progenitor cells, and isolating high-purity mononuclear cells by gradient density gradient centrifugation;
culturing the high-purity mononuclear cells overnight then purifying high-purity hematopoietic stem/progenitor cells;
incubating the high-purity hematopoietic stem/progenitor cells in IMDM/5% HABS medium supplemented with cytokines and TAT-HOXB4 for 4-7 days for expansion; and
harvesting the expanded hematopoietic stem/progenitor cells.

2. The method of claim 1, further comprising the step of:
freezing the hematopoietic stem/progenitor cells with cryoprotectant containing 24˜80% Albuminar®-25 and 20% CrySure-DEX40 containing 6˜20% human albumin.

3. The method of claim 1, wherein the high-purity mononuclear cells are incubated in a medium at a cell density of 5×10⁵ 6×10⁶ cells/mL for 16˜18 hours.

4. The method of claim 1, wherein the cytokines are IL-3, IL-6, SCF, FLT-3L or TPO.

5. The method of claim 4, wherein the cytokines comprise 5˜10 ng/mL IL-3, 10˜20 ng/mL IL-6, 50˜100 ng/mL SCF, 20˜40 ng/mL FLT-3L and 25˜50 ng/mL TPO.

6. The method of claim 1, wherein the high-purity hematopoietic stem/progenitor cells are incubated in IMDM/5% HABS medium supplemented with cytokines and TAT-HOXB4 at a cell density of 1×10⁴ 5×10⁵ cells/mL.

7. The method of claim 2, wherein the cryoprotectant comprises 80% Albuminar®-25 and 20% CrySure-DEX40 containing 20% human albumin.

8. A hematopoietic stem/progenitor cells composition prepared in accordance with the method of claim 1, comprising 15˜40% of CD34⁺CD38⁻ cells.

9. The composition of claim 8, comprising 25˜30% of CD34⁺CD38⁻ cells.

10. The method of claim 1, wherein the blood is umbilical cord blood.

11. The method of claim 1, wherein the blood is peripheral blood.
